(19) United States
(12) Patent Application Publication
(10) Pub. No.: US 2021/0395658 A1
(43) Pub. Date: Dec. 23, 2021

(54) CULTURE MEDIUM REPLACEMENT DEVICE

(71) Applicant: PHC HOLDINGS CORPORATION, Tokyo (JP)

(72) Inventors: Yoshihiro TAKAHASHI, Gunma (JP); Tomoyoshi Tokumaru, Ehime (JP)

(21) Appl. No.: 17/466,992

(22) Filed: Sep. 3, 2021

Related U.S. Application Data
(63) Continuation of application No. PCT/JP2020/005452, filed on Feb. 13, 2020.

(30) Foreign Application Priority Data
Mar. 7, 2019 (JP) 2019-041173

(51) Int. Cl. C12M 1/32

(52) U.S. Cl. CPC C12M 23/12

(57) ABSTRACT

A culture medium replacement device includes: multiple reservoirs that are mounted on a well plate including multiple wells, which each contain a culture and a culture medium, and are provided respectively for the multiple wells and that each contain a culture medium to be supplied to a corresponding well; a gas supply pump that discharges a gas supplied to the multiple reservoirs; a gas supply passage unit that connects the gas supply pump and the multiple reservoirs and that transmits the gas from the gas supply pump to the multiple reservoirs; and multiple liquid supply passages that each connect a reservoir and a corresponding well and transmit, to the corresponding well, the culture medium pushed out from the reservoir by the gas flowing into the reservoir.

### CROSS-REFERENCE TO RELATED APPLICATIONS

This application is based upon and claims the benefit of priority from the prior Japanese Patent Application No. 2019-041173, filed on Mar. 7, 2019 and International Patent Application No. PCT/JP2020/005452, filed on Feb. 13, 2020, the entire content of each of which is incorporated herein by reference.

### BACKGROUND

### Field of the Invention

The present disclosure relates to a culture medium replacement device.

### Description of the Related Art

Technologies for cultivating cells, microorganisms, and the like using a well plate with multiple wells have been conventionally known. Each well contains a culture and a culture medium. To promote multiplication of cultures or to keep the cultures in a normal state, the culture medium filled in each well needs to be regularly replaced.

With regard to the culture medium replacement, Patent Literature 1 discloses a culture medium exchange device that automatically performs culture medium replacement, for example. The culture medium exchange device includes a supply pump that supplies a culture medium into each well, a discharge pump that discharges a used culture medium from each well, multiple supply nozzles used to supply a culture medium fed from the supply pump through supply tubes into the wells, multiple on-off valves arranged between the supply pump and the supply nozzles, and multiple discharge nozzles used to discharge the culture medium from the wells. In this culture medium exchange device, the on-off valves are respectively connected between the supply pump and the supply nozzles to equalize the supply of the culture medium fed from the single supply pump to each well.
Patent Literature 1: WO2016/157378

Meanwhile, there is a constant demand for reduction of costs required for cultivation of cells or microorganisms. Accordingly, cost reduction in culture medium replacement devices is also naturally required. For the cost reduction of culture medium replacement devices, simplifying the structures of the culture medium replacement devices is desired. Such simplification of the structures of culture medium replacement devices is also desired for downsizing of the culture medium replacement devices.

### SUMMARY OF THE INVENTION

The subject application has been made in view of such a situation, and a purpose thereof is to provide a technology for simplifying the structures of culture medium replacement devices.

In response to the above issue, a culture medium replacement device according to one aspect of the subject application includes: multiple reservoirs that are mounted on a well plate including multiple wells, which each contain a culture and a culture medium, and are provided respectively for the multiple wells and that each contain a culture medium to be supplied to a corresponding well; a gas supply pump that discharges a gas supplied to the multiple reservoirs; a gas supply passage unit that connects the gas supply pump and the multiple reservoirs and that transmits the gas from the gas supply pump to the multiple reservoirs; and multiple liquid supply passages that each connect a reservoir and a corresponding well and transmit, to the corresponding well, the culture medium pushed out from the reservoir by the gas flowing into the reservoir.

A culture medium replacement device according to another aspect of the subject application includes: multiple reservoirs that are provided respectively for multiple wells, which each contain a culture and a culture medium, and that each contain a culture medium to be supplied to a corresponding well; a gas supply pump that discharges a gas supplied to the multiple reservoirs; a gas supply passage unit that connects the gas supply pump and the multiple reservoirs and that transmits the gas from the gas supply pump to the multiple reservoirs; and multiple liquid supply passages that each connect a reservoir and a corresponding well and transmit, from the reservoir toward the corresponding well, the culture medium pushed out from the reservoir by the gas flowing into the reservoir. In the gas supply passage unit, passages that respectively connect the gas supply pump and the multiple reservoirs have substantially the same volume.

Optional combinations of the aforementioned constituting elements, and implementation of the present invention, including the expressions, in the form of methods, apparatuses, or systems may also be practiced as additional modes of the present invention.

### BRIEF DESCRIPTION OF THE DRAWINGS

Embodiments will now be described, by way of example only, with reference to the accompanying drawings which are meant to be exemplary, not limiting, and wherein like elements are numbered alike in several Figures, in which:

FIG. 1 is a perspective view that illustrates a schematic structure of a cultivation apparatus that contains a well plate equipped with a culture medium replacement device according to a first embodiment;

FIG. 2 is an exploded perspective view of the culture medium replacement device;

FIG. 3 is a perspective view of the culture medium replacement device;

FIG. 4 is a perspective view of a cover;

FIGS. 5A-5D are plan views that each illustrate a layer structure of a gas supply passage unit;

FIG. 6 is a plan view that illustrates a structure of upper passages;

FIG. 7 is a plan view that illustrates a structure of top lower passages;

FIG. 8 is a plan view that illustrates a structure of middle lower passages;

FIG. 9 is a plan view that illustrates a structure of bottom lower passages;

FIG. 10 is a perspective view of multiple reservoirs and multiple liquid supply passages;

FIG. 11 is a sectional end view of the multiple reservoirs and the multiple liquid supply passages;

FIG. 12 is a diagram that shows a mechanism of culture medium supply from the multiple reservoirs to multiple wells;

FIG. 13 is a perspective view of some of multiple liquid drainages;

FIG. 14 is a diagram that shows a mechanism of culture medium collection from the multiple wells to a liquid drain tank; and

FIGS. 15A-15C are plan views that each illustrate a layer structure of a gas supply passage unit included in a culture medium replacement device according to a second embodiment.

### DETAILED DESCRIPTION OF THE INVENTION

In the following, the present invention will be described based on preferred embodiments with reference to the drawings. The embodiments are intended to be illustrative only and not to limit the invention, so that it should be understood that not all of the features or combinations thereof described in the embodiments are necessarily essential to the invention. Like reference characters denote like or corresponding constituting elements, members, and processes in each drawing, and repetitive description will be omitted as appropriate.

The scale or shape of each component shown in each drawing is defined for the sake of convenience to facilitate the explanation and is not to be regarded as limitative unless otherwise specified. Also, when the terms “first”, “second”, and the like are used in the present specification or claims, such terms do not imply any order or degree of importance and are used to distinguish one configuration from another, unless otherwise specified. Further, in each drawing, part of a member less important in describing embodiments may be omitted.

### First Embodiment

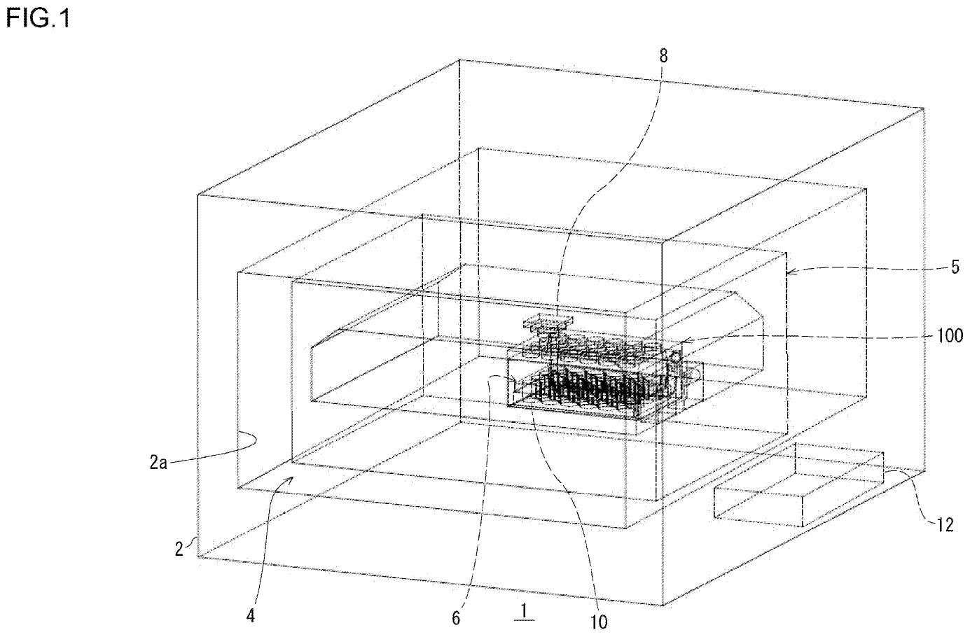

FIG. 1 is a perspective view that illustrates a schematic structure of a cultivation apparatus that contains a well plate equipped with a culture medium replacement device according to the first embodiment. A cultivation apparatus 1 may be a CO₂ incubator, for example, and includes an insulated box body 2 having an opening 2a on the front thereof, and a door (not illustrated) that covers the opening 2a to be openable and closable. Within the insulated box body 2, a cultivation chamber 4 is provided. The cultivation chamber 4 contains an analysis device 5 that has an observation function. A well plate 6 and a culture medium replacement device 100 attached thereto are contained in the analysis device 5. For example, the culture medium replacement device 100 may be set in the analysis device 5 having an observation function or in another observation device to be used.

The analysis device 5 includes a camera 8 having an observation function. The camera 8 is disposed above the well plate 6 and fixed to be able to capture an image of the well plate 6. The well plate 6 includes multiple wells 10 that each contain a culture and a culture medium. The multiple wells 10 are horizontally arranged in a matrix. The camera 8 may be moved to a position above each well 10 of the well plate 6 to capture an image of the culture within the well 10. The cultures cultivated in the well plate 6 may be cells, for example.

In the insulated box body 2, a control device 12 is provided. The control device 12 may be implemented by an element such as a CPU or memory of a computer or by a circuit as a hardware configuration, and by a computer program or the like as a software configuration. The control device 12 receives a signal from an external device connected to the cultivation apparatus 1 via a network or from an operation unit (not illustrated) provided in the cultivation apparatus 1, for example. Based on the signal thus received, the control device 12 controls the operation of each unit in the cultivation apparatus 1. For example, the control device 12 performs adjustment of the temperature and humidity within the cultivation chamber 4. The control device 12 also controls the operation of the analysis device 5, such as driving of the camera 8. The analysis device 5 may include a control unit, separately from the control device 12, to control the driving of the analysis device 5 itself. The control device 12 also transmits a signal for ordering culture medium replacement to the culture medium replacement device 100.

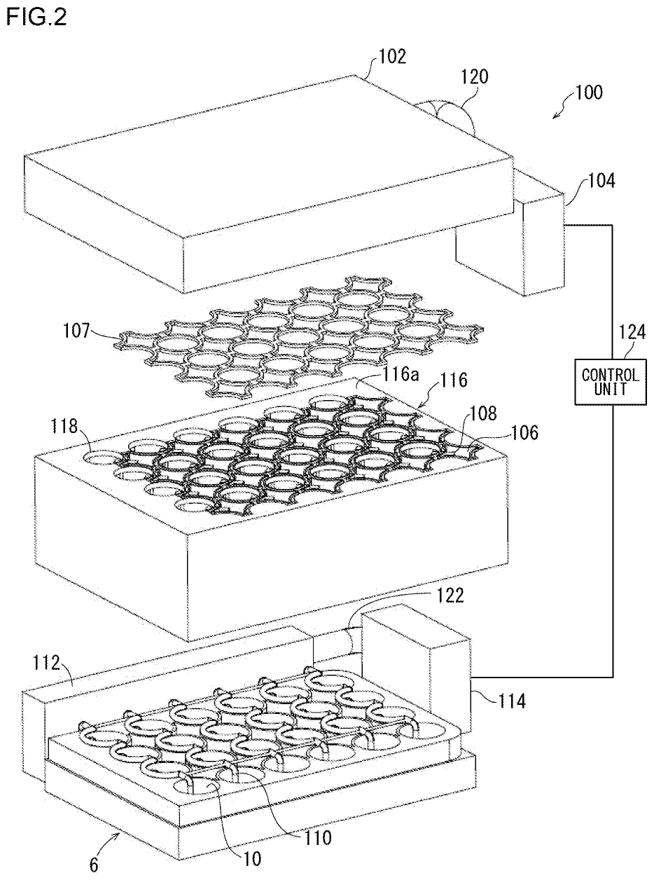

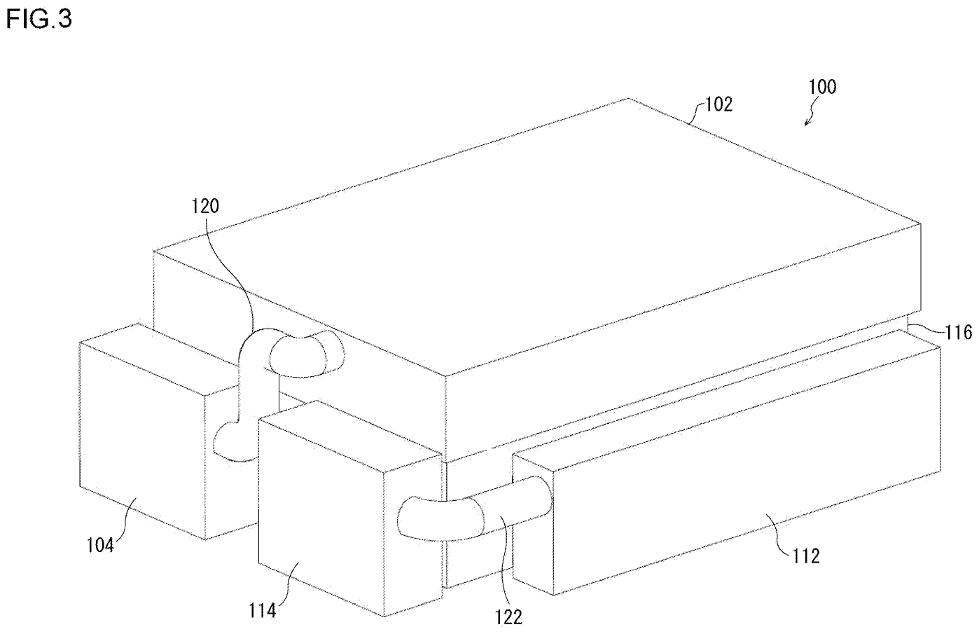

In the following, the culture medium replacement device 100 will be described in detail. FIG. 2 is an exploded perspective view of the culture medium replacement device 100. FIG. 3 is a perspective view of the culture medium replacement device 100. In FIG. 2, a control unit 124 is illustrated as a functional block. As with the control device 12, this functional block may be implemented by an element such as a CPU or memory of a computer or by a circuit as a hardware configuration, and by a computer program or the like as a software configuration. It will be understood by those skilled in the art that these functional blocks may be implemented in a variety of forms by combinations of hardware and software.

The culture medium replacement device 100 includes a cover 102, a gas supply pump 104, multiple reservoirs 106, a seal member 107, multiple liquid supply passages 108, multiple liquid drainages 110, a liquid drain tank 112, and a suction pump 114.

The cover 102, the multiple reservoirs 106, the multiple liquid supply passages 108, and the multiple liquid drainages 110 may be made of resin, for example. The multiple reservoirs 106 are mounted on the well plate 6. In other words, the multiple reservoirs 106 are arranged within a range where the well plate 6 extends, when viewed from a vertical direction. In the present embodiment, the multiple reservoirs 106 are provided in an inner lid member 116. The inner lid member 116 is almost box-shaped and includes, on a top surface 116a thereof, multiple recesses arranged in a matrix, which constitute the multiple reservoirs 106. The inner lid member 116 covers the well plate 6 from above. Also, the cover 102 covers the inner lid member 116 from above. Between the cover 102 and the inner lid member 116, the seal member 107 is disposed.

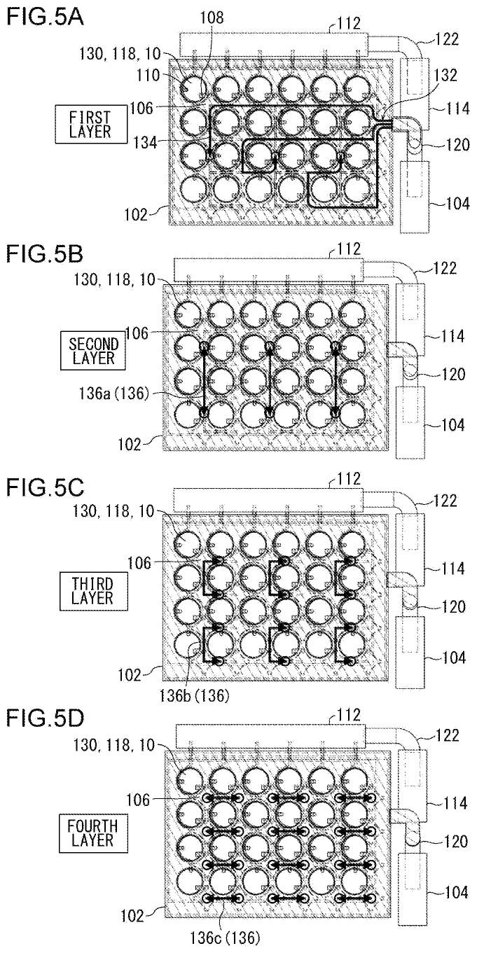

On the top surface 116a of the inner lid member 116, multiple openings 118 are provided. The multiple openings 118 are arranged such as to overlap with the respective wells 10 when viewed from a vertical direction, in the state where the inner lid member 116 is attached to the well plate 6 (see FIG. 5A, for example). The well plate 6 of the present embodiment is a microplate with 24 wells, as an example. Accordingly, the inner lid member 116 has 24 openings 118. The number of wells in the well plate 6 may be 6, 96, 384, or the like. Also, a commercially available product may be used for the well plate 6.

The multiple reservoirs 106 are arranged to be shifted in a horizontal direction with respect to the multiple openings 118 and the multiple wells 10 (see FIG. 5A, for example). Each well 10 and each opening 118 in the present embodiment is circular when viewed from a vertical direction. Meanwhile, each reservoir 106 is substantially rhombic and is disposed in a space among the openings 118 arranged in a matrix. The multiple reservoirs 106 are provided such as to respectively correspond to the multiple wells 10 on a one-to-one basis. Accordingly, the inner lid member 116 in the present embodiment includes 24 reservoirs 106. Each reservoir 106 contains a culture medium, which is supplied to a corresponding well 10 at the time of culture medium replacement. The culture medium within each reservoir 106 is supplied to a corresponding well 10 via a liquid supply passage 108 that connects the reservoir 106 and the well 10. The structures of the reservoirs 106 and the liquid supply passages 108 will be detailed later.

The amount of the culture medium contained in each reservoir 106 may be an amount that can be used in a single culture medium replacement process, for example. When the culture medium within each reservoir 106 is entirely supplied to a well 10 in a single culture medium replacement process, the amount of the culture medium supplied to each well 10 can be equalized more accurately. Alternatively, each reservoir 106 may be sectioned into multiple rooms that each contain an amount of the culture medium that can be used in a single culture medium replacement process, and each room and a corresponding well 10 may be connected by a liquid supply passage 108. In this case, when the inner lid member 116 is placed once, the culture medium replacement process can be performed multiple times. Alternatively, an amount of the culture medium required to perform the culture medium replacement process multiple times may be filled in each reservoir 106, and the culture medium within each reservoir 106 may be gradually supplied to a well 10 in each culture medium replacement process. In other words, the culture medium within each reservoir 106 may be supplied to a well 10 divisionally in multiple times.

The gas supply pump 104 may be disposed along a side surface of the cover 102 and a side surface of the inner lid member 116, for example. The gas supply pump 104 is a device that discharges a gas supplied to the multiple reservoirs 106. The gas discharged from the gas supply pump 104 may be atmospheric gas within the cultivation chamber 4, for example. For the gas supply pump 104, a publicly-known pump may be used. The gas supply pump 104 is connected to the cover 102 via a gas supply duct 120. The number of gas supply pump 104 is smaller than the number of reservoirs 106. In the present embodiment, a single gas supply pump 104 supplies the gas to the 24 reservoirs 106.

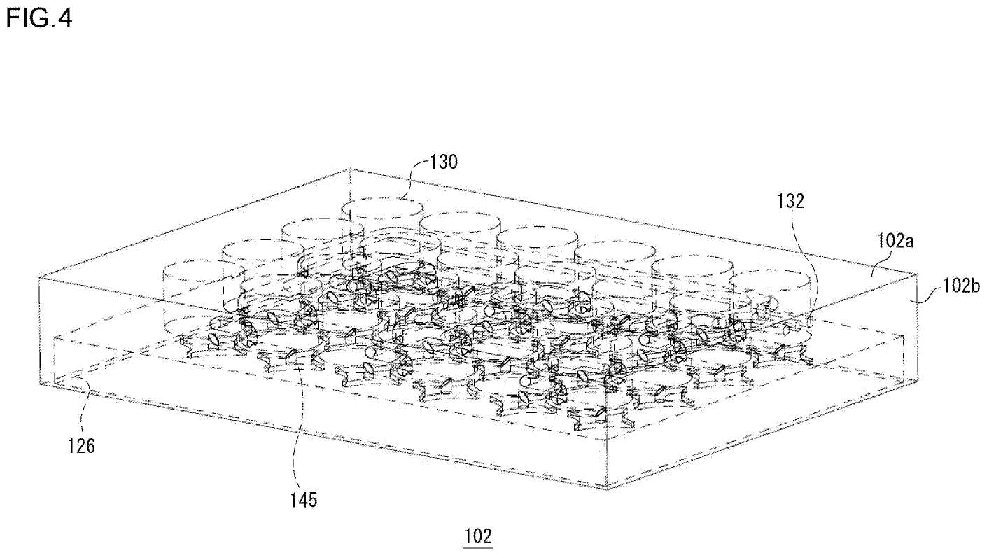

The gas discharged from the gas supply pump 104 is sent to a gas supply passage unit 132 (see FIG. 4) within the cover 102 via the gas supply duct 120. The gas is then supplied to each reservoir 106 through the gas supply passage unit 132. The structure of the gas supply passage unit 132 will be detailed later.

The multiple liquid drainages 110 are pipes that connect the multiple wells 10 and the suction pump 114 and transmit the culture medium from each well 10 to the suction pump 114 side. The multiple liquid drainages 110 are provided such as to respectively correspond to the multiple wells 10 on a one-to-one basis. Each liquid drainage 110 is inserted at one end into a well 10 and connected at the other end to the liquid drain tank 112. Each liquid drainage 110 in the present embodiment is partially provided in common with other liquid drainages 110.

The liquid drain tank 112 is a container that stores the culture medium that has flown through the liquid drainages 110. The liquid drain tank 112 is disposed along a side surface of the cover 102 and a side surface of the inner lid member 116, for example. The liquid drain tank 112 of the present embodiment is disposed along a side surface of the cover 102 and a side surface of the inner lid member 116 that intersect with the side surfaces along which the gas supply pump 104 is disposed. To the liquid drain tank 112, the suction pump 114 is connected via a gas intake duct 122. Accordingly, the liquid drain tank 112 is connected between the suction pump 114 and the multiple liquid drainages 110.

The suction pump 114 may be disposed alongside of the gas supply pump 104 along the side surfaces of the cover 102 and the inner lid member 116, for example. The suction pump 114 is a device that suctions the used culture medium from the multiple wells 10. For the suction pump 114, a publicly-known pump may be used. The number of suction pump 114 is smaller than the number of wells 10. In the present embodiment, a single suction pump 114 suctions the culture medium from the 24 wells 10. When the suction pump 114 is driven, the culture medium in each well 10 is drawn into a liquid drainage 110, moved toward the liquid drain tank 112 through the liquid drainage 110, and collected into the liquid drain tank 112. The structure of the liquid drainages 110 and the mechanism of collecting the used culture medium will be detailed later.

The culture medium replacement device 100 includes the control unit 124. The control unit 124 is constituted by a control substrate, which is fixed to a side surface of the cover 102 or the inner lid member 116, for example. The control unit 124 is connected to the control device 12 of the cultivation apparatus 1 to receive, from the control device 12, a signal for ordering culture medium replacement. Upon reception of the signal for ordering culture medium replacement, the control unit 124 transmits a drive signal to the gas supply pump 104 and the suction pump 114 only during a predetermined period of time. Accordingly, the gas supply pump 104 and the suction pump 114 operate only while receiving the drive signal. For example, after the suction pump 114 is driven and the used culture medium in each well 10 is collected, the gas supply pump 104 is driven and the unused culture medium in each reservoir 106 is supplied to a corresponding well 10. The drive time of the gas supply pump 104 is a time required to complete the supply of the unused culture medium. Also, the drive time of the suction pump 114 is a time required to complete the collection of the used culture medium.

A user of the cultivation apparatus 1 can set a timing of culture medium replacement by means of an external device connected to the cultivation apparatus 1 via a network or by means of an operation unit provided in the cultivation apparatus 1. Based on the timing setting, the control device 12 transmits the signal for ordering culture medium replacement to the control unit 124. Also, the control unit 124 may include a timer for measuring the elapse of time. Accordingly, the user of the cultivation apparatus 1 may set, in the timer, the time until culture medium replacement is performed, so that the control unit 124 executes the culture medium replacement. In this case, when the timer detects the elapse of the time set by the user, the control unit 124 transmits the drive signal to the gas supply pump 104 and the suction pump 114. The control unit 124 may receive the signal for ordering culture medium replacement directly from an external device connected to the cultivation apparatus 1 via a network or the like, without the intervention of the control device 12.

In the following, the configuration of each unit of the culture medium replacement device 100 will be described in detail. First, the cover 102 will be described. FIG. 4 is a perspective view of the cover 102. In FIG. 4, the internal structure of the cover 102 is indicated by dotted lines. The cover 102 is a substantially box-shaped member having a top surface 102a of a rectangular shape, and four side surfaces 102b that extend downward from the respective sides of the top surface 102a. The cover 102 includes an opening 126 that has a shape extending along the outer shape of the inner lid member 116 and that is positioned opposite to the top surface 102a in a vertical direction. The opening 126 is defined by the lower sides of the four side surfaces 102b.

Also, on the top surface 102a, multiple observation windows 130 are provided. The multiple observation windows 130 are arranged such as to overlap with the respective wells 10 when viewed from a vertical direction, in the state where the cover 102 is attached to the well plate 6. Accordingly, when viewed from a vertical direction, each observation window 130, each opening 118 of the inner lid member 116, and each well 10 overlap with one another. In the cover 102, at least the observation windows 130 are transparent. Accordingly, through the observation windows 130, the state within each well 10 can be observed and captured using the camera 8. The cover 102 in the present embodiment is entirely made of a transparent resin material. Also, in the cover 102, the observation windows 130 are made thinner than the other portions.

In the top surface 102a, the gas supply passage unit 132 is provided. The gas supply passage unit 132 is a pipe unit that connects the gas supply pump 104 and the multiple reservoirs 106 and that transmits the gas from the gas supply pump 104 to the multiple reservoirs 106. FIGS. 5A-5D are plan views that each illustrate a layer structure of the gas supply passage unit 132. FIG. 5A illustrates passages in the first layer, FIG. 5B illustrates passages in the second layer, FIG. 5C illustrates passages in the third layer, and FIG. 5D illustrates passages in the fourth layer. The passages in the first through fourth layers are laminated in this order from the top in the vertical direction.

The gas supply passage unit 132 includes upper passages 134 into which the gas from the gas supply pump 104 flows, and multiple lower passages 136 that are branched from the upper passages 134 to be connected to the respective reservoirs 106 and that transmit the gas from the upper passages 134 to the respective reservoirs 106. The gas supply passage unit 132 in the present embodiment has a four-layer structure. The upper passages 134 correspond to the passages of the gas supply passage unit 132 in the first layer. The multiple lower passages 136 correspond to the passages of the gas supply passage unit 132 in the second through fourth layers. In the following description, the lower passages 136 in the second layer will be referred to as top lower passages 136a, the lower passages 136 in the third layer will be referred to as middle lower passages 136b, and the lower passages 136 in the fourth layer will be referred to as bottom lower passages 136c. The upper passages 134 are connected to the top lower passages 136a, which are connected to the middle lower passages 136b, which are connected to the bottom lower passages 136c. The bottom lower passages 136c are connected to the respective reservoirs 106.

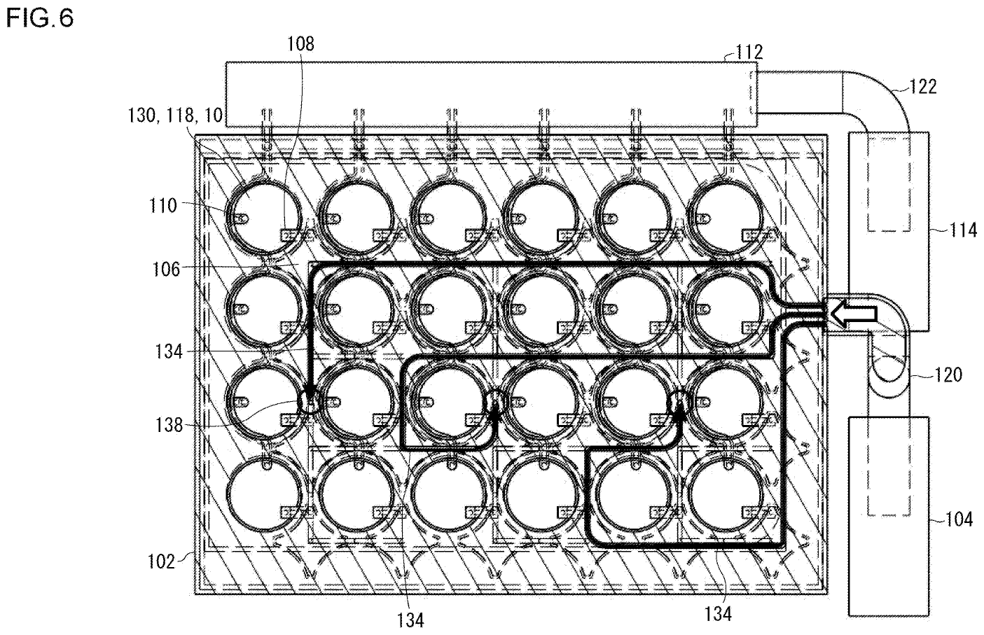

FIG. 6 is a plan view that illustrates the structure of the upper passages 134. The gas supply passage unit 132 includes the upper passages 134 smaller in number than the reservoirs 106. The gas supply passage unit 132 in the present embodiment includes three upper passages 134. One end of each upper passage 134 is connected to the gas supply duct 120. Each upper passage 134 is routed in horizontal directions within the top surface 102a to reach a connection part 138 with a top lower passage 136a. The other end of each upper passage 134 is connected to a top lower passage 136a at a connection part 138.

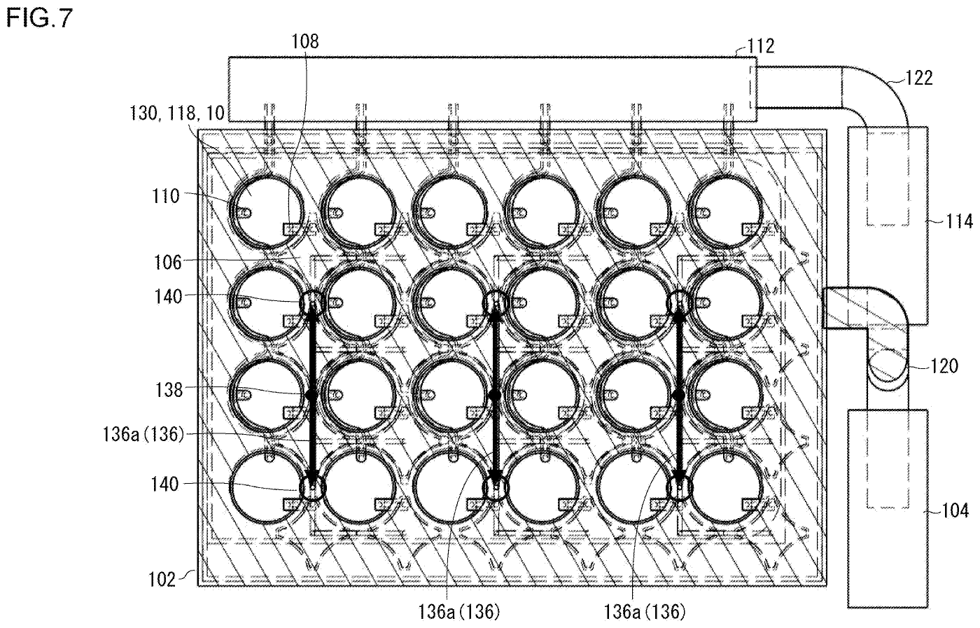

FIG. 7 is a plan view that illustrates the structure of the top lower passages 136a. The gas supply passage unit 132 in the present embodiment includes three top lower passages 136a. Each top lower passage 136a extends in two directions from a connection part 138 with an upper passage 134 and is routed in horizontal directions within the top surface 102a to reach connection parts 140 with middle lower passages 136b. The two end parts of each top lower passage 136a are respectively connected to middle lower passages 136b at connection parts 140.

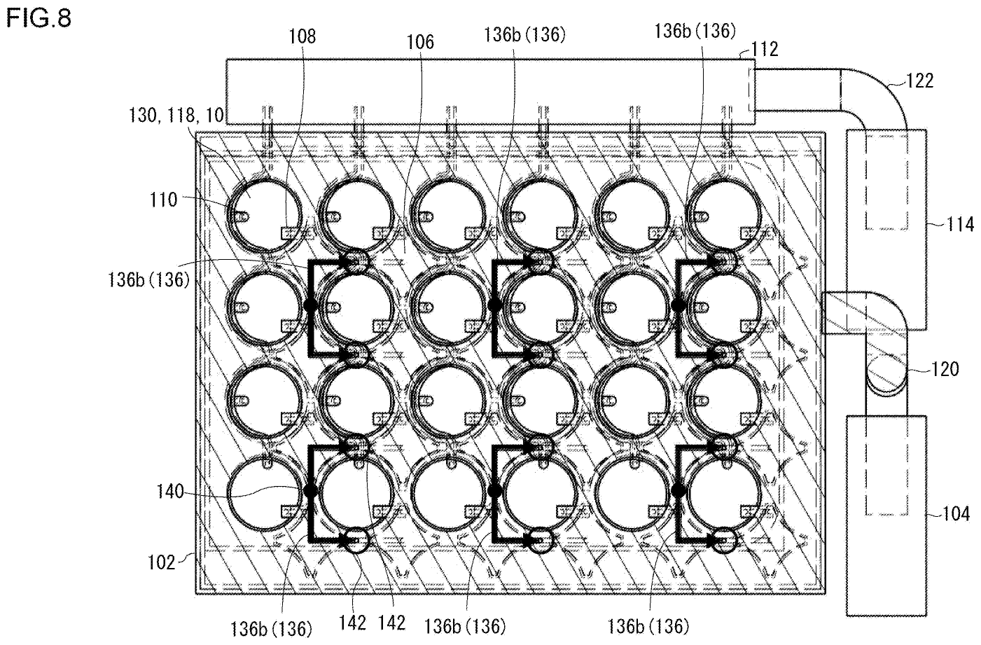

FIG. 8 is a plan view that illustrates the structure of the middle lower passages 136b. The gas supply passage unit 132 in the present embodiment includes six middle lower passages 136b. Each middle lower passages 136b extends in two directions from a connection part 140 with a top lower passage 136a and is routed in horizontal directions within the top surface 102a to reach connection parts 142 with bottom lower passages 136c. The two end parts of each middle lower passage 136b are respectively connected to bottom lower passages 136c at connection parts 142.

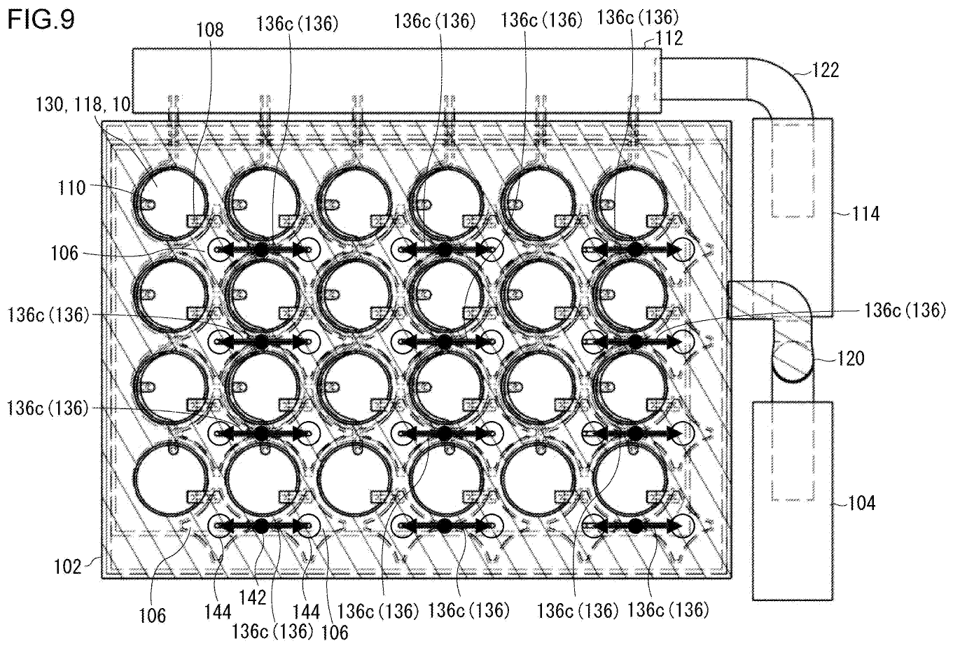

FIG. 9 is a plan view that illustrates the structure of the bottom lower passages 136c. The gas supply passage unit 132 in the present embodiment includes twelve bottom lower passages 136c. Each bottom lower passage 136c extends in two directions from a connection part 142 with a middle lower passage 136b and is routed in horizontal directions within the top surface 102a to reach corresponding reservoirs 106. The two end parts of each bottom lower passage 136c constitute gas outlets 144, which are connected to corresponding reservoirs 106.

Thus, the number of flows of gas discharged from the gas supply pump 104 increases to the number corresponding to that of the reservoirs 106 while the gas flows from the upper passages 134, which are smaller in number than the reservoirs 106, through the top lower passages 136a, the middle lower passages 136b, and the bottom lower passages 136c. The gas flow in the present embodiment is branched into 3 in the upper passages 134 in the first layer, branched into 6 in the top lower passages 136a in the second layer, branched into 12 in the middle lower passages 136b in the third layer, and then branched into 24, which is equal to the number of reservoirs 106, in the bottom lower passages 136c in the fourth layer.

The gas supply passage unit 132 is disposed to be shifted in a horizontal direction with respect to the multiple wells 10. In the present embodiment, each of the upper passages 134 and the lower passages 136 is provided between observation windows 130. The “being shifted in a horizontal direction” means that each passage in the gas supply passage unit 132 does not overlap with the center of a well 10 when viewed from a vertical direction. It is suitable that, when viewed from a vertical direction, each passage in the gas supply passage unit 132 does not overlap with a region that corresponds to 80% of the whole area of a well 10 and of which the center coincides with the center of the well 10 (hereinafter, the region will be referred to as the “80% region”). It is more suitable that, when viewed from a vertical direction, each passage in the gas supply passage unit 132 does not overlap with any part of the entirety of a well 10.

The center of a well 10 viewed from a vertical direction may be the geometrical center of the shape of the well 10 viewed from a vertical direction, for example. Accordingly, when the state within each well 10 is captured using the camera 8, interference by the gas supply passage unit 132 with the capturing can be prevented. Each passage in the gas supply passage unit 132 of the present embodiment is disposed such as not to overlap with any part of the entirety of a well 10 when viewed from a vertical direction.

The cover 102 is fixed to the inner lid member 116 by means of a fixation mechanism, such as snap fitting. The cover 102 is fixed to the inner lid member 116 with the seal member 107 provided between the cover 102 and the inner lid member 116 (see FIG. 2). The seal member 107 may be a gasket made of rubber, for example, and has a structure in which frames that each have the same shape as the upper opening of each reservoir 106 are arranged in the same way as the multiple reservoirs 106 and integrally formed. With the cover 102 fixed to the inner lid member 116, the seal member 107 hermetically connects the upper openings of the multiple reservoirs 106 and the gas outlets 144 of the gas supply passage unit 132. Thus, each reservoir 106 is hermetically sealed. This can restrain leakage of the gas transmitted from the gas supply passage unit 132 to the reservoirs 106, through a space between the cover 102 and the inner lid member 116 to the outside.

Also, as illustrated in FIG. 4, the cover 102 includes a projection 145 around each gas outlet 144 of the gas supply passage unit 132. Each projection 145 has the same frame shape as the upper opening of each reservoir 106 and projects from a surface of the top surface 102a that faces the inner lid member 116, toward the inner lid member 116 side. With the cover 102 fixed to the inner lid member 116, the tip of each projection 145 enters a corresponding reservoir 106. This can improve the airtightness of the reservoirs 106. Also, when the cover 102 is attached to the inner lid member 116, the projections 145 serve the function of positioning the cover 102 and the inner lid member 116.

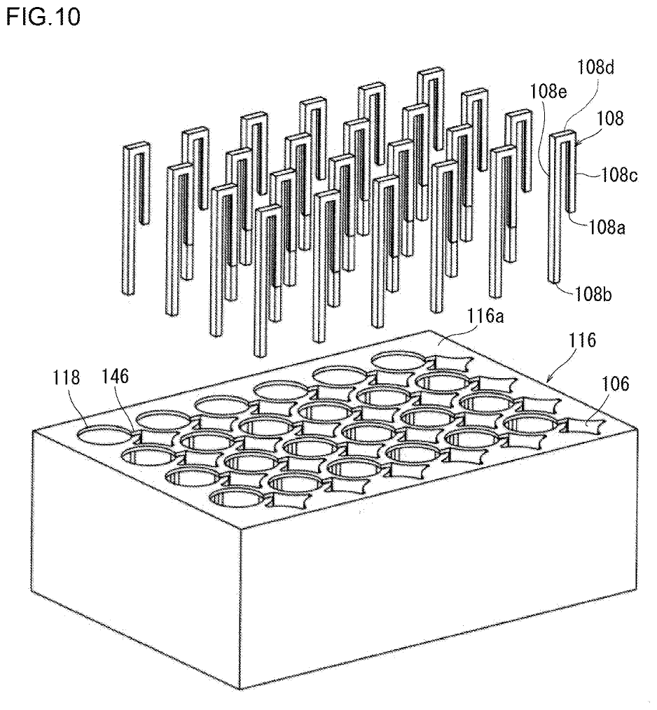

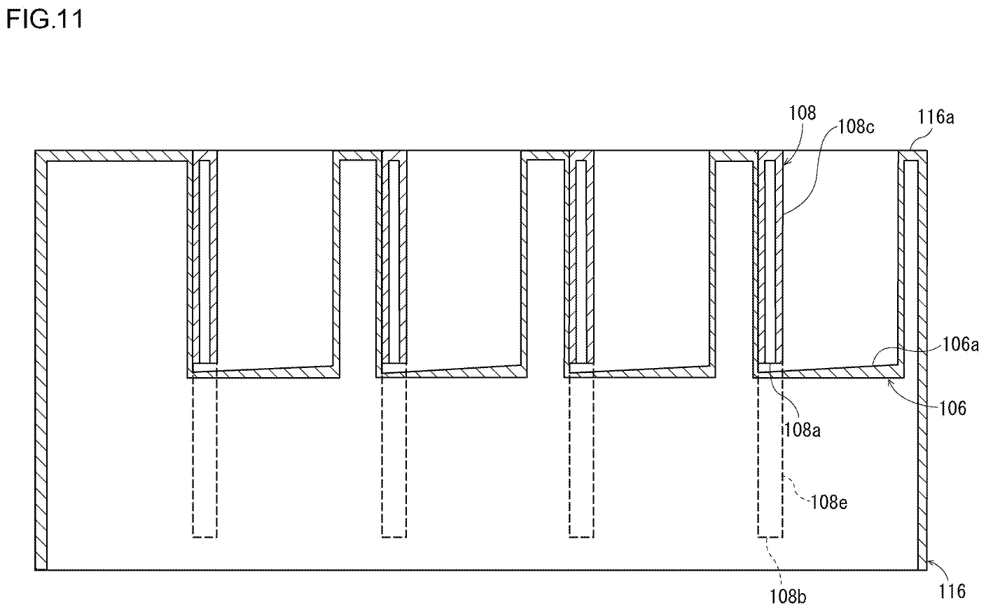

There will now be described the multiple reservoirs 106 and the multiple liquid supply passages 108. FIG. 10 is a perspective view of the multiple reservoirs 106 and the multiple liquid supply passages 108. FIG. 11 is a sectional end view of the multiple reservoirs 106 and the multiple liquid supply passages 108.

As described previously, in the present embodiment, the multiple reservoirs 106 are constituted by the multiple recesses provided on the top surface 116a of the inner lid member 116. Also, on the top surface 116a, the multiple openings 118 are provided. The multiple openings 118 are arranged such as to overlap with the respective wells 10 when viewed from a vertical direction, and the multiple reservoirs 106 are arranged to be shifted in a horizontal direction with respect to the multiple wells 10 when viewed from a vertical direction (see FIG. 5A, for example).

The “being shifted in a horizontal direction” means that each reservoir 106 does not overlap with the center of a well 10 when viewed from a vertical direction. It is suitable that, when viewed from a vertical direction, each reservoir 106 does not overlap with the 80% region of a well 10. It is more suitable that, when viewed from a vertical direction, each reservoir 106 does not overlap with any part of the entirety of a well 10. Accordingly, when the state within each well 10 is captured using the camera 8, interference by the reservoirs 106 with the capturing can be prevented. Each reservoir 106 in the present embodiment is disposed such as not to overlap with any part of the entirety of a well 10 when viewed from a vertical direction.

Each liquid supply passage 108 is a pipe that connects a reservoir 106 and a well 10 and that transmits the culture medium within the reservoir 106 to the well 10. Each liquid supply passage 108 includes a first end part 108a, a second end part 108b, a first pipe part 108c, a second pipe part 108d, and a third pipe part 108e.

In each liquid supply passage 108, the first end part 108a is inserted into a reservoir 106. The first pipe part 108c is connected at the lower end to the first end part 108a and extends upward from the first end part 108a along a wall surface of the reservoir 106. The second pipe part 108d is connected at one end to the upper end of the first pipe part 108c and extends horizontally to cross a boundary part 146 between the reservoir 106 and an opening 118. The third pipe part 108e is connected at the upper end to the other end of the second pipe part 108d and extends downward to be connected to the second end part 108b. The second end part 108b is inserted into a well 10. The boundary part 146 has a shape recessed downward. The second pipe part 108d is fitted into the recessed part. Thus, the liquid supply passages 108 are fixed to the inner lid member 116.

Each reservoir 106 has a bottom surface 106a tilted to be lower toward the first end part 108a of the liquid supply passage 108 connected to the reservoir 106. With such a bottom surface 106a, the culture medium within the reservoir 106 can be led to the first end part 108a side by means of gravity. Accordingly, the culture medium within each reservoir 106 can be transferred to a well 10 more certainly, and the amount of the culture medium in each well 10 can be equalized more accurately.

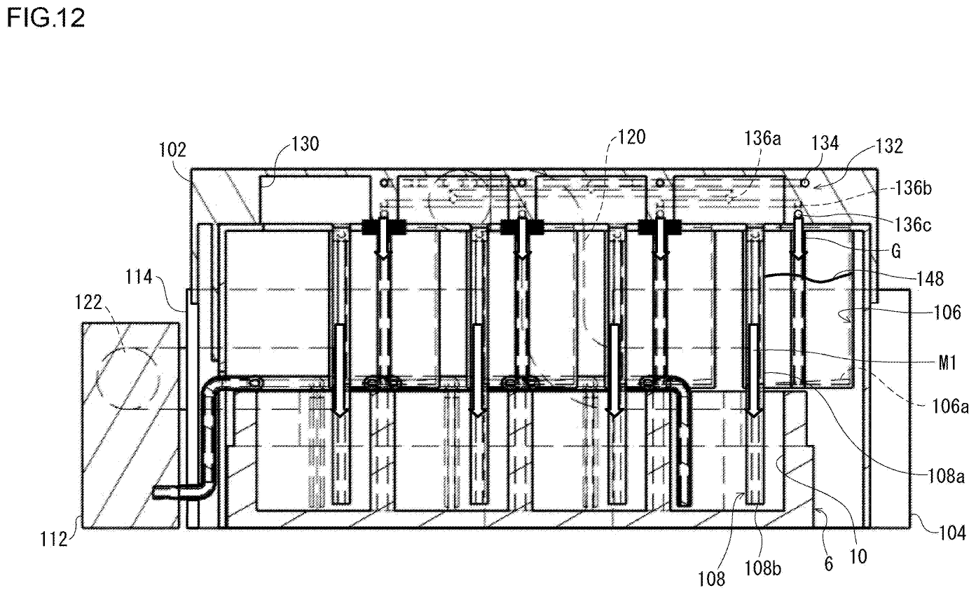

There will now be described the mechanism of culture medium supply from the reservoirs 106 to the wells 10. FIG. 12 shows a mechanism of culture medium supply from the multiple reservoirs 106 to the multiple wells 10. As an example, a culture medium is dispensed into each reservoir 106 in advance using a pipette or the like. Thereafter, the cover 102 is attached to the inner lid member 116. With the cover 102 fixed to the inner lid member 116, each reservoir 106 is hermetically closed with the seal member 107. Thereafter, the inner lid member 116 with the cover 102 is attached to the well plate 6. For the culture medium replacement, a gas is discharged from the gas supply pump 104 and flows through the gas supply duct 120 and the gas supply passage unit 132, so as to flow from the bottom lower passages 136c into the respective reservoirs 106 (the flows indicated by arrows G). When the gas flows into each reservoir 106, the pressure within the reservoir 106 becomes positive.

Accordingly, a culture medium 148 within each reservoir 106 is pushed toward the bottom surface 106a of the reservoir 106 and flows into a liquid supply passage 108 through the first end part 108a. Each liquid supply passage 108 transmits, to a corresponding well 10, the culture medium 148 pushed out from a corresponding reservoir 106 by the gas flowing into the reservoir 106 (the flows indicated by arrows M1). Thus, the culture medium 148 within each reservoir 106 is supplied to a corresponding well 10.

In the gas supply passage unit 132, the passages that respectively connect the gas supply pump 104 and the multiple reservoirs 106 have substantially the same volume. In other words, each gas passage from the end part connected to the gas supply duct 120 to the end part connected to a reservoir 106 has substantially the same volume.

As an example, in the present embodiment, the shape of the twelve bottom lower passages 136c is determined such that each of the passages from a connection part 142 with a middle lower passage 136b to two gas outlets 144 has the same volume. Subsequently, the shape of the six middle lower passages 136b is determined such that each of the passages from a connection part 140 with a top lower passage 136a to two connection parts 142 has the same volume. Subsequently, the shape of the three top lower passages 136a is determined such that each of the passages from a connection part 138 with an upper passage 134 to two connection parts 140 has the same volume. Thereafter, the laying route of each upper passage 134 from the gas supply duct 120 to a corresponding connection part 138 is determined such that the three upper passages 134 connected respectively to the three connection parts 138 have the same volume.

If the amount of gas supplied to each reservoir 106 is not equal, the termination time of culture medium supply in each well 10 could be different. More specifically, the culture medium supply from a reservoir 106 to which a larger amount of gas is supplied will be terminated earlier than that from a reservoir 106 to which a smaller amount of gas is supplied. With the passage structure in which the gas passages are branched from the upper passages 134 to the bottom lower passages 136c, i.e., with the structure in which each gas passage for a corresponding well 10 is partially provided in common with other gas passages, if there is a difference in termination time of culture medium supply, the gas to be supplied to a reservoir 106 in which the culture medium supply has not been completed will preferentially flow into a reservoir 106 in which the culture medium supply has been completed. This is because the reservoir 106 in which the culture medium supply has been completed does not contain the culture medium 148 that provides gas flow resistance, so that the gas smoothly flows through the reservoir 106. In this case, the culture medium supply may not be completed in some reservoirs 106, and the amount of the culture medium in each well 10 could be different.

Meanwhile, when the passages connecting the gas supply pump 104 and the respective reservoirs 106 have substantially the same volume, the amount of gas supplied to each reservoir 106 can be equalized. As a result, the amount of the culture medium in each well 10 can be equalized more accurately. For example, a difference in amount of culture medium in each reservoir 106 can be made to be 5% or less. The “being substantially the same” suitably means that each passage volume is completely the same but may include a case where there is a difference in passage volume that causes an admissible difference in growing condition of the culture in each well 10. Further, the “being the same” may also be construed to include the case where there is a difference in passage volume that causes an admissible difference in growing condition of the culture in each well 10, in addition to the case where each passage volume is completely the same.

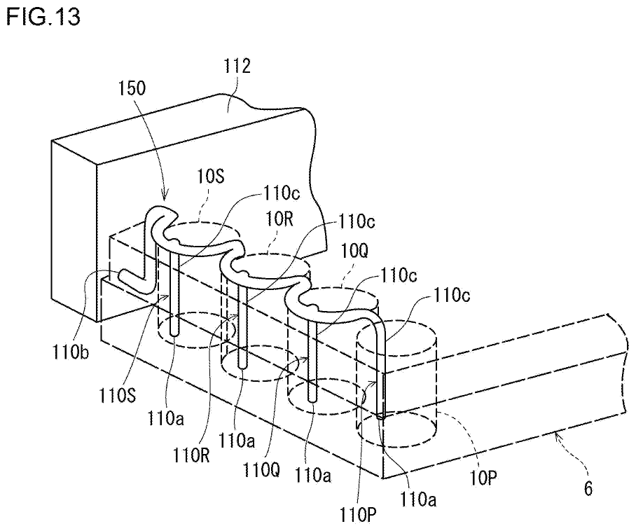

There will now be described the structure of the multiple liquid drainages 110 and the mechanism of culture medium collection from the wells 10. FIG. 13 is a perspective view of some of the multiple liquid drainages 110. Each liquid drainage 110 includes a first end part 110a, a second end part 110b, and a pipe part 110c. In each liquid drainage 110, the first end part 110a is inserted into a well 10. The pipe part 110c is connected at one end to the first end part 110a and extends upward along a wall surface of the well 10; upon reaching the upper end part of the well 10, the pipe part 110c extends horizontally toward the liquid drain tank 112. The other end part of the pipe part 110c is connected to the second end part 110b, which is connected to the liquid drain tank 112.

The multiple liquid drainages 110 in the present embodiment are grouped into multiple aggregates 150. Each aggregate 150 is constituted by a predetermined number of liquid drainages 110. In each aggregate 150, each liquid drainage 110 is partially provided in common with other liquid drainages 110. For example, considering multiple wells 10 arranged in a 4-by-6 matrix, liquid drainages 110 for the four wells 10 in each column constitute an aggregate 150. Accordingly, the multiple liquid drainages 110 are grouped into six aggregates 150, which each are constituted by four liquid drainages 110 (see also FIG. 2).

The liquid drain tank 112 extends in a row direction of the well plate 6. In the following description, the four wells 10 in each column will be referred to as a first well 10P, a second well 10Q, a third well 10R, and a fourth well 10S, from the side farther from the liquid drain tank 112, for the sake of convenience. Also, a liquid drainage 110 of which the first end part 110a is inserted into the first well 10P will be referred to as a first liquid drainage 110P, a liquid drainage 110 of which the first end part 110a is inserted into the second well 10Q will be referred to as a second liquid drainage 110Q, a liquid drainage 110 of which the first end part 110a is inserted into the third well 10R will be referred to as a third liquid drainage 110R, and a liquid drainage 110 of which the first end part 110a is inserted into the fourth well 10S will be referred to as a fourth liquid drainage 110S.

The pipe part 110c of the first liquid drainage 110P extends upward from the first end part 110a along a wall surface of the first well 10P and then extends horizontally from the upper end part of the first well 10P toward the liquid drain tank 112. The pipe part 110c extends along the edges of the second well 10Q, the third well 10R, and the fourth well 10S, i.e., curvedly extends to be diverted around the second well 10Q through the fourth well 10S when viewed from a vertical direction, to be connected to the second end part 110b. Thus, part of each liquid drainage 110 is disposed to be shifted in a horizontal direction with respect to the multiple wells 10.

For example, a portion of each pipe part 110c extending along the upper surface of the well plate 6 is disposed to be shifted in a horizontal direction with respect to the multiple wells 10. The “being shifted in a horizontal direction” means the same as defined for the gas supply passage unit 132. In the present embodiment, such a portion is provided in a region that is closer to an outer circumference of each well 10 and that corresponds to 20% of the whole area of each well 10 when viewed from a vertical direction. In other words, each pipe part 110c is disposed such as not to overlap with the 80% region of each well 10.

The pipe part 110c of the second liquid drainage 110Q extends upward along a wall surface of the second well 10Q and merges into the pipe part 110c of the first liquid drainage 110P at the upper end part of the second well 10Q. The pipe part 110c then extends along the edges of the third well 10R and the fourth well 10S to be connected to the second end part 110b. Thus, in the pipe part 110c of the first liquid drainage 110P, the portion from the connection part with the pipe part 110c of the second liquid drainage 110Q to the liquid drain tank 112 side also serves as the pipe part 110c of the second liquid drainage 110Q.

The pipe part 110c of the third liquid drainage 110R extends upward along a wall surface of the third well 10R and merges into the pipe part 110c of the first liquid drainage 110P at the upper end part of the third well 10R. The pipe part 110c then extends along the edge of the fourth well 10S to be connected to the second end part 110b. Thus, in the pipe part 110c of the first liquid drainage 110P, the portion from the connection part with the pipe part 110c of the third liquid drainage 110R to the liquid drain tank 112 side also serves as the pipe part 110c of the third liquid drainage 110R. Also, the pipe part 110c into which the pipe part 110c of the third liquid drainage 110R merges also corresponds to the pipe part 110c of the second liquid drainage 110Q. Accordingly, it can also be said that, in the pipe part 110c of the second liquid drainage 110Q, the portion from the connection part with the pipe part 110c of the third liquid drainage 110R to the liquid drain tank 112 side also serves as the pipe part 110c of the third liquid drainage 110R.

The pipe part 110c of the fourth liquid drainage 110S extends upward along a wall surface of the fourth well 10S and merges into the pipe part 110c of the first liquid drainage 110P at the upper end part of the fourth well 10S. The pipe part 110c then extends toward the liquid drain tank 112 to be connected to the second end part 110b. Thus, in the pipe part 110c of the first liquid drainage 110P, the portion from the connection part with the pipe part 110c of the fourth liquid drainage 110S to the liquid drain tank 112 side also serves as the pipe part 110c of the fourth liquid drainage 110S. Also, the pipe part 110c into which the pipe part 110c of the fourth liquid drainage 110S merges also corresponds to the pipe parts 110c of the second liquid drainage 110Q and the third liquid drainage 110R. Accordingly, it can also be said that, in the pipe parts 110c of the second liquid drainage 110Q and the third liquid drainage 110R, the portion from the connection part with the pipe part 110c of the fourth liquid drainage 110S to the liquid drain tank 112 side also serves as the pipe part 110c of the fourth liquid drainage 110S.

The second end part 110b is provided in common for the first liquid drainage 110P through the fourth liquid drainage 110S, and the culture medium 148 within the first well 10P through the fourth well 10S flows into the liquid drain tank 112 through the same second end part 110b. In other words, each aggregate 150 in the present embodiment is constituted by a main pipe and branch pipes that are branched from the main pipe to be inserted respectively into the first well 10P through the fourth well 10S. The main pipe is connected at the base end part to the liquid drain tank 112 and extends along the upper edges of the fourth well 10S, the third well 10R, and the second well 10Q such that the tip part of the main pipe extends to the first well 10P. Also, from midway along the main pipe, branch pipes extend respectively into the fourth well 10S, the third well 10R, and the second well 10Q, and the other branch pipe extends from the tip part of the main pipe into the first well 10P.

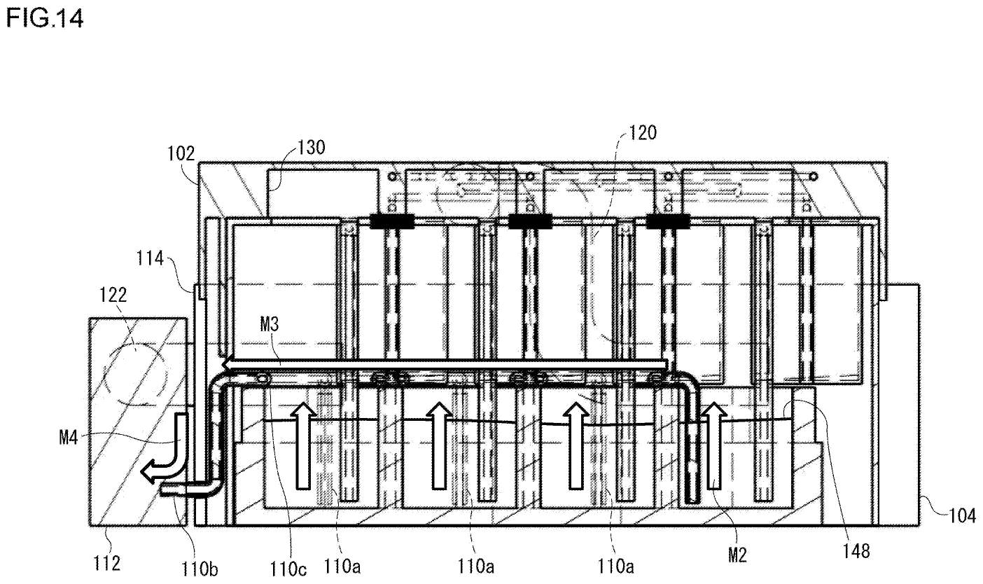

FIG. 14 shows a mechanism of culture medium collection from the multiple wells 10 to the liquid drain tank 112. With the suction pump 114 driven, the culture medium 148 within each well 10 is collected into the liquid drain tank 112. The suction pump 114 in the present embodiment suctions the atmospheric gas within the liquid drain tank 112. This makes the pressure within the liquid drain tank 112 negative, so that the culture medium 148 in each well 10 is drawn into a corresponding liquid drainage 110 that connects the liquid drain tank 112 and the well 10 (the flows indicated by arrows M2).

The culture medium 148 in each well 10 flows through a pipe part 110c toward the liquid drain tank 112 (the flow indicated by an arrow M3) and flows into the liquid drain tank 112 through the second end part 110b (the flow indicated by an arrow M4). Since the culture medium 148 is trapped in the liquid drain tank 112, the gas intake duct 122 and the suction pump 114 are not made dirty with the culture medium 148. Accordingly, the maintainability of the culture medium replacement device 100 can be improved. Alternatively, the liquid drain tank 112 may be omitted, and the culture medium 148 may be drawn into the suction pump 114.

Similarly, since the gas supply pump 104 discharges the atmospheric gas toward each reservoir 106, the gas supply pump 104 and the gas supply duct 120 are also not made dirty with the culture medium 148. Also in this regard, the maintainability of the culture medium replacement device 100 can be improved. In addition, the same gas supply pump 104, gas supply duct 120, suction pump 114, and gas intake duct 122 can be used for multiple times of cultivation. Accordingly, increase in cultivation cost can be restrained.

As an example of usage modes of the culture medium replacement device 100, the gas supply pump 104, the gas supply duct 120, the suction pump 114, the gas intake duct 122, and the liquid drain tank 112 may be installed on a slide tray (not illustrated) provided within the cultivation chamber 4. The cover 102 is detachably connected to the gas supply duct 120, and the multiple liquid drainages 110 are detachably connected to the liquid drain tank 112. Accordingly, a user of the culture medium replacement device 100 can install or remove only the well plate 6, the inner lid member 116, and the cover 102 in or from the cultivation chamber 4.

For example, the user may put the inner lid member 116, which contains the culture medium 148 for replacement, and the cover 102 over the well plate 6, in which a culture is disseminated and a culture medium is added, to integrate the components and may then place the integrated components on the tray taken out of the cultivation chamber 4. Thereafter, the user connects the gas supply duct 120 and the cover 102, connects the liquid drain tank 112 and the liquid drainages 110, and returns the tray into the cultivation chamber 4. Accordingly, the culture can be cultivated while culture medium replacement is automatically performed by means of the culture medium replacement device 100. Also, the state of the culture can be observed and captured using the camera 8.

The multiple reservoirs 106, the multiple liquid supply passages 108, and the multiple liquid drainages 110 are suitably formed integrally. More specifically, each liquid supply passage 108 is fitted into a boundary part 146 of the inner lid member 116 in which the multiple reservoirs 106 are formed, so that the multiple reservoirs 106 and the multiple liquid supply passages 108 are integrally formed. Also, the inner lid member 116 includes a support part (not illustrated) for liquid drainages 110, and the multiple liquid drainages 110 are attached to the support part, so that the multiple reservoirs 106, the multiple liquid supply passages 108, and the multiple liquid drainages 110 are integrally formed.

Thus, when a user of the culture medium replacement device 100 only puts the inner lid member 116 over the well plate 6, the reservoirs 106 and the wells 10 can be respectively connected by the liquid supply passages 108, and the liquid drainages 110 can be respectively inserted into the wells 10. Also, the gas supply passage unit 132 is integrally provided with the cover 102. Accordingly, only by placing the cover 102 over the inner lid member 116, the gas supply passage unit 132 and the reservoirs 106 can be connected.

As described above, the culture medium replacement device 100 according to the present embodiment includes: the multiple reservoirs 106 that are mounted on the well plate 6 including the multiple wells 10 and are provided for the respective wells 10 and that each contain a culture medium to be supplied to a corresponding well 10; the gas supply pump 104 that discharges a gas supplied to the multiple reservoirs 106; the gas supply passage unit 132 that connects the gas supply pump 104 and the multiple reservoirs 106 and that transmits the gas from the gas supply pump 104 to the multiple reservoirs 106; and the multiple liquid supply passages 108 that each connect a reservoir 106 and a corresponding well 10 and transmit, to the corresponding well 10, the culture medium 148 pushed out from the reservoir 106 by the gas flowing into the reservoir 106.

Thus, in the present embodiment, the culture medium 148 for replacement is distributed to each reservoir 106, and, with the gas supplied from the gas supply pump 104, the culture medium 148 is pushed out from the reservoir 106 into a corresponding well 10. Accordingly, by adjusting the amount of the culture medium 148 distributed to each reservoir 106, the amount of the culture medium supplied to each well 10 can be equalized. Therefore, the structure of the culture medium replacement device can be simplified, compared to a conventional culture medium replacement device in which the amount of the culture medium fed from the supply pump is controlled with an on-off valve connected to each supply nozzle. This enables downsizing of the culture medium replacement device 100.

Also, since the multiple reservoirs 106 are mounted on the well plate 6, the area required for installation of the culture medium replacement device 100 can be reduced. In other words, the culture medium replacement device 100 can be downsized. In addition, by downsizing the culture medium replacement device 100, the culture medium replacement device 100 can be easily built into an analysis device with an observation function or into another observation device provided within the cultivation apparatus 1. The culture medium replacement device 100 can be also easily built into an analysis device with an incubator function and an observation function or into an observation device with an incubator function.

The multiple reservoirs 106 are arranged to be shifted in a horizontal direction with respect to the multiple wells 10. This can ensure the view field of the camera 8. Also, the number of gas supply pump 104 is smaller than the number of reservoirs 106, and the gas discharged from the gas supply pump 104 is distributed to each reservoir 106 through the gas supply passage unit 132. Accordingly, compared to the case where the gas supply pumps 104 are provided for the respective reservoirs 106 on a one-to-one basis, the necessary parts can be reduced. Therefore, the culture medium replacement device 100 can be structured more simply and can also be downsized.

The gas supply passage unit 132 includes the upper passages 134 into which the gas from the gas supply pump 104 flows and of which the number is smaller than that of the reservoirs 106, and the multiple lower passages 136 that are branched from the upper passages 134 to be connected to the respective reservoirs 106. With such a branch structure of the gas supply passage unit 132 from the upstream side toward the downstream side, the structure of the entire gas supply passage unit 132 can be simplified, compared to the case where the gas supply pump 104 and the respective reservoirs 106 are connected by multiple separate gas supply passages. Also, the area required for laying of the gas supply passage unit 132 can be reduced. Therefore, the culture medium replacement device 100 can be structured more simply and can also be downsized.

The gas supply passage unit 132 is disposed to be shifted in a horizontal direction with respect to the multiple wells 10. Accordingly, the gas supply pump 104 and each reservoir 106 can be connected while the view field of the camera 8 is ensured. Also, when the gas supply passage unit 132 is laid to be diverted around the wells 10, such laying can be easily achieved with the branch structure of the gas supply passage unit 132.

In the gas supply passage unit 132, the passages connecting the gas supply pump 104 and the respective reservoirs 106 have substantially the same volume. Accordingly, the amount of the culture medium 148 supplied from each reservoir 106 to a corresponding well 10 can be equalized. Also, when each passage volume is made substantially the same, the branch structure of the gas supply passage unit 132 can restrain significant increase in laying area of the entire gas supply passage unit 132. Thus, both making each passage volume substantially the same and ensuring the view field of the camera 8 can be achieved.

Each reservoir 106 has the bottom surface 106a tilted to be lower toward the first end part 108a of the liquid supply passage 108 connected to the reservoir 106. Accordingly, the culture medium 148 within the reservoirs 106 can be supplied to the wells 10 more certainly, and remaining of the culture medium 148 within the wells 10 can be restrained. Therefore, the amount of the culture medium 148 supplied to each well 10 can be equalized more certainly. The culture medium replacement device 100 also includes the seal member 107 that hermetically connects the multiple reservoirs 106 and the gas supply passage unit 132. Accordingly, the amount of the culture medium 148 supplied to each well 10 can be equalized more certainly.

The culture medium replacement device 100 also includes the suction pump 114 that suctions the culture medium 148 from the multiple wells 10, and the multiple liquid drainages 110 that connect the multiple wells 10 and the suction pump 114 and transmit the culture medium 148 flowing from each well 10 toward the suction pump 114. Thus, with the structure in which a single suction pump 114 suctions the culture medium 148 from the multiple wells 10, the necessary parts can be reduced, compared to the case where the suction pumps 114 are provided for the respective wells 10 on a one-to-one basis. Therefore, the culture medium replacement device 100 can be structured more simply and can also be downsized.

The culture medium replacement device 100 also includes the liquid drain tank 112 that is connected between the suction pump 114 and the multiple liquid drainages 110 and that stores the culture medium 148 that has flown through the liquid drainages 110. Accordingly, contact between the suction pump 114 and the culture medium 148 can be avoided. Therefore, the maintainability of the culture medium replacement device 100 can be improved. Also, since the suction pump 114 can be used for multiple times of cultivation, increase in cultivation cost can be restrained.

The culture medium replacement device 100 of the present embodiment has a structure in which the inner lid member 116 and the cover 102 are placed over the well plate 6, the gas supply duct 120 is connected to the cover 102, and the liquid drain tank 112 is connected to the inner lid member 116 (the liquid drainage 110 thereof). Accordingly, without modifying the well plate 6 of a commercially available product, the culture medium replacement device 100 can be attached. Therefore, a culture medium replacement device with favorable usability can be provided.

### Second Embodiment

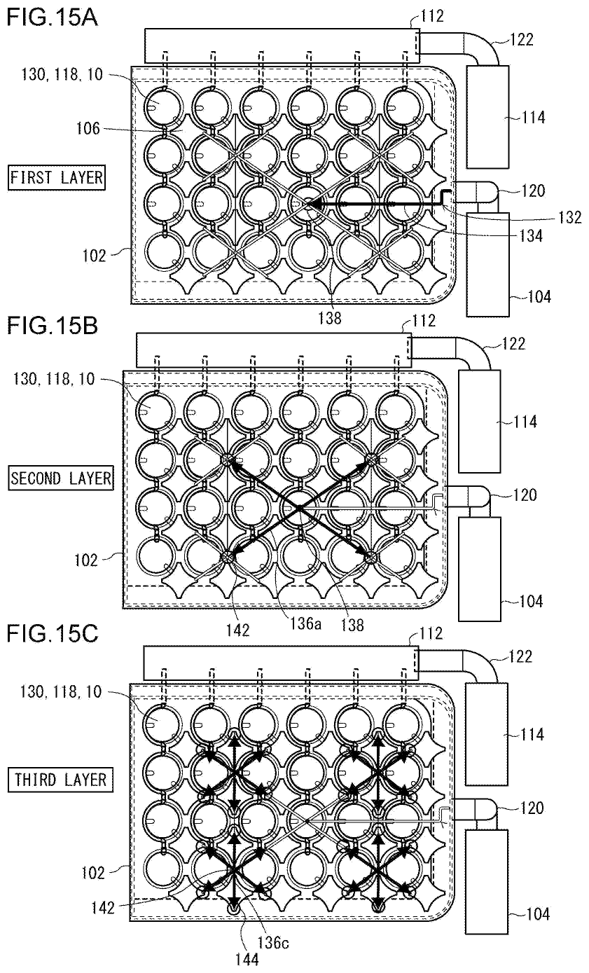

A culture medium replacement device according to the second embodiment includes configurations in common with the culture medium replacement device in the first embodiment, except the structure of the gas supply passage unit 132. In the description of the present embodiment, the description regarding the configurations similar to those in the first embodiment will be omitted as appropriate. FIGS. 15A-15C are plan views that each illustrate a layer structure of the gas supply passage unit 132 included in the culture medium replacement device 100 according to the second embodiment. FIG. 15A illustrates a passage in the first layer, FIG. 15B illustrates a passage in the second layer, and FIG. 15C illustrates passages in the third layer. The passages in the first through third layers are laminated in this order from the top in the vertical direction.

The gas supply passage unit 132 includes an upper passage 134 into which the gas from the gas supply pump 104 flows, and multiple lower passages 136 that are branched from the upper passage 134 to be connected to the respective reservoirs 106. The gas supply passage unit 132 in the present embodiment has a three-layer structure.

The upper passage 134 correspond to the passage of the gas supply passage unit 132 in the first layer. The multiple lower passages 136 correspond to the passages of the gas supply passage unit 132 in the second and third layers. In the following description, a lower passage 136 in the second layer will be referred to as a top lower passage 136a, and lower passages 136 in the third layer will be referred to as bottom lower passages 136c. The upper passage 134 is connected to the top lower passage 136a, which is connected to the bottom lower passages 136c, which are connected to the respective reservoirs 106.

The gas supply passage unit 132 includes the upper passage 134 smaller in number than the reservoirs 106. The gas supply passage unit 132 in the present embodiment includes one upper passage 134. One end of the upper passage 134 is connected to the gas supply duct 120. The upper passage 134 is routed in horizontal directions within the top surface 102a to reach a connection part 138 with a top lower passage 136a. The other end of the upper passage 134 is connected to the top lower passage 136a at the connection part 138. The connection part 138 is provided at a position that overlaps with the center of a predetermined well 10.

The gas supply passage unit 132 in the present embodiment includes one top lower passage 136a. The top lower passage 136a extends in four directions from the connection part 138 with the upper passage 134 and is routed in horizontal directions within the top surface 102a to reach connection parts 142 with bottom lower passages 136c. The four end parts of the top lower passage 136a are respectively connected to the bottom lower passages 136c at the connection parts 142.

The gas supply passage unit 132 in the present embodiment includes four bottom lower passages 136c. Each bottom lower passage 136c extends in six directions from a connection part 142 with the top lower passage 136a and is routed in horizontal directions within the top surface 102a to reach corresponding reservoirs 106. The six end parts of each bottom lower passage 136c constitute gas outlets 144 that are connected to corresponding reservoirs 106.

Accordingly, in the present embodiment, there is only one gas passage in the upper passage 134 in the first layer, which is branched into 4 in the top lower passage 136a in the second layer, which are then branched into 24, which is equal to the number of reservoirs 106, in the bottom lower passages 136c in the third layer. Also with such a structure of the gas supply passage unit 132, the structure of the entire gas supply passage unit 132 can be simplified, compared to the case where the gas supply pump 104 and the respective reservoirs 106 are connected by multiple separate gas supply passages. Also, the area required for laying of the entire gas supply passage unit 132 can be reduced. Therefore, the culture medium replacement device 100 can be structured more simply and can also be downsized.

Also in the present embodiment, the volume of each gas passage can be made substantially the same. Further, while the substantially the same passage volume is maintained, the gas supply passage unit 132 can be disposed to be shifted in a horizontal direction with respect to multiple wells 10 except one well 10 that overlaps with the connection part 138. Accordingly, when the state within each well 10 is captured using the camera 8, interference by the gas supply passage unit 132 with the capturing can be prevented.

Although the upper passage 134 in FIG. 15A is provided such as to pass through the centers of two wells 10 positioned between the gas supply duct 120 and the connection part 138, the upper passage 134 may be diverted such as not to overlap with the two wells 10. Also, although the top lower passage 136a in FIG. 15B linearly extends from the connection part 138 toward the connection parts 142, the top lower passage 136a may be diverted such as not to overlap with the wells 10. Similarly, although the bottom lower passages 136c in FIG. 15C linearly extend from the connection parts 142 toward the gas outlets 144, the bottom lower passages 136c may be diverted such as not to overlap with the wells 10.

Exemplary embodiments of the present invention have been described in detail. Each of the abovementioned embodiments merely describes a specific example for carrying out the present invention. The embodiments are not intended to limit the technical scope of the present invention, and various design modifications, including changes, addition, and deletion of constituting elements, may be made to the embodiments without departing from the scope of ideas of the invention defined in the claims. Such an additional embodiment with a design modification added has the effect of each of the combined embodiments and modifications. In the aforementioned embodiments, matters to which design modifications may be made are emphasized with the expression of “of the present embodiment”, “in the present embodiment”, or the like, but design modifications may also be made to matters without such expression. Optional combinations of the abovementioned constituting elements may also be employed as additional aspects of the present invention. Also, the hatching provided on the cross sections in the drawings is not provided to limit the materials of the objects with the hatching.

The embodiments may be defined by the following item.

A culture medium replacement device (100), including:
multiple reservoirs (106) provided respectively for multiple wells (10) that each contain a culture and a culture medium (148), the multiple reservoirs (106) each containing a culture medium (148) to be supplied to a corresponding well (10);
a gas supply pump (104) that discharges a gas supplied to the multiple reservoirs (106);
a gas supply passage unit (132) that connects the gas supply pump (104) and the multiple reservoirs (106) and that transmits the gas from the gas supply pump (104) to the multiple reservoirs (106); and
multiple liquid supply passages (108) that each connect a reservoir (106) and a corresponding well (10) and transmit, from the reservoir (106) toward the corresponding well (10), the culture medium (148) pushed out from the reservoir (106) by the gas flowing into the reservoir (106), wherein,
in the gas supply passage unit (132), passages that respectively connect the gas supply pump (104) and the multiple reservoirs (106) have substantially the same volume.

According to this aspect, the amount of the culture medium supplied to each reservoir 106 can be equalized without providing an on-off valve for each reservoir 106. Therefore, the culture medium replacement device 100 can be structured more simply and can also be downsized.

What is claimed is:

1. A culture medium replacement device, comprising:
a plurality of reservoirs mounted on a well plate including a plurality of wells that each contain a culture and a culture medium and provided respectively for the plurality of wells, the plurality of reservoirs each containing a culture medium to be supplied to a corresponding well;
a gas supply pump that discharges a gas supplied to the plurality of reservoirs;
a gas supply passage unit that connects the gas supply pump and the plurality of reservoirs and that transmits the gas from the gas supply pump to the plurality of reservoirs; and
a plurality of liquid supply passages that each connect a reservoir and a corresponding well and transmit, to the corresponding well, the culture medium pushed out from the reservoir by the gas flowing into the reservoir.

2. The culture medium replacement device according to claim 1, wherein the plurality of reservoirs are arranged to be shifted in a horizontal direction with respect to the plurality of wells.

3. The culture medium replacement device according to claim 1, wherein the number of the gas supply pump is smaller than the number of the reservoirs.

4. The culture medium replacement device according to claim 3, wherein the gas supply passage unit comprises:
an upper passage into which a gas from the gas supply pump flows and of which the number is smaller than that of the reservoirs; and
a plurality of lower passages branched from the upper passage to be connected respectively to the reservoirs.

5. The culture medium replacement device according to claim 1, wherein, in the gas supply passage unit, passages that respectively connect the gas supply pump and the plurality of reservoirs have substantially the same volume.

6. The culture medium replacement device according to claim 1, wherein each of the reservoirs has a bottom surface tilted to be lower toward an end part of a corresponding one of the liquid supply passages connected to the reservoir.

7. The culture medium replacement device according to claim 1, wherein the gas supply passage unit is disposed to be shifted in a horizontal direction with respect to the plurality of wells.

8. The culture medium replacement device according to claim 1, further comprising a seal member that hermetically connects the plurality of reservoirs and the gas supply passage unit.

9. The culture medium replacement device according to claim 1, further comprising:
a suction pump that suctions a culture medium from each of the plurality of wells; and
a plurality of liquid drainages that connect the plurality of wells and the suction pump and that transmit the culture medium from each of the plurality of wells to the suction pump side.

10. The culture medium replacement device according to claim 9, further comprising a liquid drain tank that is connected between the suction pump and the plurality of liquid drainages and that stores the culture medium that has flown through the liquid drainages.

11. A culture medium replacement device, comprising:
a plurality of reservoirs provided respectively for a plurality of wells that each contain a culture and a culture medium, the plurality of reservoirs each containing a culture medium to be supplied to a corresponding well;
a gas supply pump that discharges a gas supplied to the plurality of reservoirs;
a gas supply passage unit that connects the gas supply pump and the plurality of reservoirs and that transmits the gas from the gas supply pump toward the plurality of reservoirs; and
a plurality of liquid supply passages that each connect a reservoir and a corresponding well and transmit, from the reservoir toward the corresponding well, the culture medium pushed out from the reservoir by the gas flowing into the reservoir, wherein,
in the gas supply passage unit, passages that respectively connect the gas supply pump and the plurality of reservoirs have substantially the same volume.
